(19) United States
(12) Patent Application Publication
(10) Pub. No.: US 2026/0207842 A1
(43) Pub. Date: Jul. 23, 2026

(54) KEEP VEIN OPEN INFUSION CONTROL DEVICE

(71) Applicant: CareFusion 303, Inc., San Diego, CA (US)

(72) Inventors: Praveen NALAWADE, Bengaluru (IN); Rahul Rajan PUTHUKKAD, Bengaluru (IN); Narsi Reddy SANIKOMMU, Kanigiri (IN); Greeshma G, Thiruvananthapuram (IN); Kowshika K, Tirupur (IN)

(21) Appl. No.: 19/035,523

(22) Filed: Jan. 23, 2025

(51) Int. Cl. A61M 5/145; A61L 29/04; A61M 39/10; A61M 5/14

(52) U.S. Cl. CPC A61M 5/145; A61L 29/041; A61M 39/10; A61M 2005/1404; A61M 2005/14513; A61M 2039/1038; A61M 2202/0007; A61M 2202/04

(57) ABSTRACT

In order to maintain the patency of an intravenous (IV) catheter while limiting the risk of occlusions, it can be desirable to use a keep vein open (KVO) infusion flow control device to continuously deliver fluid to a patient’s catheter line in a controlled manner. The KVO infusion flow control device disclosed herein utilizes super absorbent polymers (SAPs), which expand when in contact with water (particularly, distilled water), to gradually deliver a consistent flow of saline to the patient’s catheter line. The KVO infusion flow control device includes a spiking system that can be used to spike a distilled water chamber and expose distilled water to a SAP block. When the SAP block expands, the SAP block compresses a flush chamber filled with saline and advances the saline out of the KVO infusion flow control device and into the patient’s catheter line.

### TECHNICAL FIELD

The present invention relates generally to keep vein open (KVO) (also referred to as "to keep open" or "TKO") devices and, in particular, to a KVO device for administrating a medical fluid, such as saline, a therapeutic agent, and/or total parenteral nutritional fluid, to a patient as a continuous, substantially continuous, semi-continuous, or intermittent fluid flow.

### BACKGROUND

Medical treatments often include the infusion of a medical fluid (e.g., a saline solution or a liquid medication) to patients using an intravenous (IV) catheter. In situations where a patient might need to receive medication through infusion on a repeated or intermittent basis, it can be desirable to maintain the patency of the IV catheter by preventing the patient's vein from closing. However, there is a challenge in keeping the unused catheter lumens patent because catheter lines may tend to occlude due to various biological responses in the patient over long periods of time between active flow in the lumens. For example, blood clots can form inside the patient's blood vessels, or the catheter line can be occluded with precipitates from drugs or parenteral nutrition. In addition to inconveniencing caregivers, occlusions in IV lines can increase the risk of infections in the patient's bloodstream by creating nesting environments for microorganisms.

### SUMMARY

Rinsing peripheral intravenous catheters (PIVCs) is a common means for maintaining catheter patency. Particularly, rinsing PIVCs is thought to prevent occlusion of the inner lumen of the catheter line (in addition to reducing the formation of blood clots, bacterial biofilm, and drug precipitates). However, existing solutions for preventing occlusions in IV catheter lines are not ideal because a clinician has to flush the unused lumen(s) with saline and/or multiple IV drip bags, even if the patient is already receiving the one IV medication infusion. Therefore, a KVO infusion flow control device is desirable for providing safe and controlled fluid flow. By providing a slow, continuous infusion of fluid (e.g., saline) to the patient, the patient's vein can be kept open while avoiding occlusions in the catheter line.

Some embodiments of the present disclosure are directed to a keep vein open (KVO) infusion control device, the KVO infusion control device comprising: a housing comprising a tube having a tip at a distal end of the tube and a spiking system at a proximal portion of the tube; a flush chamber disposed within a distal portion of the housing, the flush chamber configured to be filled with saline; a super absorbent polymer (SAP) block disposed within the housing and proximal to the flush chamber, the SAP block configured to expand when in contact with distilled water; and a distilled water chamber disposed within the housing adjacent to the spiking system and proximal to the SAP block, the distilled water chamber configured to be filled with the distilled water, wherein the spiking system is configured to facilitate delivery of the distilled water to the SAP block, wherein the SAP block is configured to compress the flush chamber and distally advance the saline out of the tip when the SAP block expands.

In some embodiments, the tip is configured to couple with a needleless fluid connector. In some embodiments, the tip comprises a male luer lock.

In some embodiments, the SAP block is generally cylindrical. In some embodiments, the SAP block comprises sodium polyacrylate. In some embodiments, the SAP block comprises poly(acrylamide-co-acrylic acid) potassium salt.

In some embodiments, the KVO infusion control device further comprises a uniform pusher positioned between the SAP block and the flush chamber. Optionally, the uniform pusher is configured to receive a force from the SAP block as the SAP block expands and apply the force to the flush chamber to distally advance the saline out of the tip.

In some embodiments, the distilled water chamber is configured to be refilled with the distilled water via an opening at a proximal end of the distilled water chamber.

In some embodiments, the KVO infusion control device further comprises a cap coupled to a proximal end of the housing, wherein the cap is configured to close the distilled water chamber.

In some embodiments, the spiking system comprises one or more spikes positioned on an inner surface of the housing, each of the one or more spikes comprising a sharp end configured to puncture the distilled water chamber to fluidly connect the distilled water chamber to the housing. Optionally, the spiking system comprises one or more spikes configured to puncture the distilled water chamber and fluidly connect the distilled water chamber to the SAP block. Optionally, the spiking system is configured to puncture the distilled water chamber and release the distilled water into the housing.

Some embodiments of the present disclosure are directed to a keep vein open (KVO) infusion control device, the KVO infusion control device comprising: a housing comprising a tube having a distal tip; a flush chamber disposed within a distal portion of the housing, the flush chamber configured to be filled with saline; a super absorbent polymer (SAP) block disposed within the housing and proximal to the flush chamber, the SAP block configured to expand when in contact with distilled water; a distilled water chamber disposed within the housing and proximal to the SAP block, the distilled water chamber configured to be filled with the distilled water; and a spiking system positioned on an inner surface of the housing and proximal to the distilled water chamber, the spiking system configured to fluidly connect the distilled water chamber to the housing and facilitate delivery of the distilled water to the SAP block, wherein the SAP block is configured to compress the flush chamber and distally advance the saline out of the distal tip

In some embodiments, the KVO infusion control device further comprises a cap coupled to a proximal end of the housing, wherein the cap is configured to close the distilled water chamber.

In some embodiments, the spiking system is attached to the cap and configured to puncture the distilled water chamber when the cap is coupled to the proximal end of the housing. Optionally, the spiking system comprises one or more spikes, each of the one or more spikes comprising a distally oriented sharp end configured to puncture the distilled water chamber to fluidly connect the distilled water chamber to the housing.

Other embodiments of the present disclosure are directed to a keep vein open (KVO) infusion control device, the KVO infusion control device comprising: a housing comprising a tube having a tip at a distal end of the tube and a spiking system at a proximal portion of the tube; a flush chamber disposed within a distal portion of the housing and configured to be filled with saline, the flush chamber comprising a super absorbent polymer (SAP) tube that is configured to expand when in contact with distilled water; and a distilled water chamber disposed within the housing adjacent to the spiking system and proximal to the flush chamber, the distilled water chamber configured to be filled with the distilled water, wherein the spiking system is configured to facilitate delivery of the distilled water to the SAP tube, wherein the SAP tube is configured to displace the saline out of the tip when the SAP tube expands.

In some embodiments, the spiking system is configured to puncture the distilled water chamber and release the distilled water into the housing so that the distilled water can contact the SAP tube.

In some embodiments, when in contact with the distilled water, the SAP tube is configured to expand radially inward and displace the saline out of the tip.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates a KVO infusion control device being used in combination with an IV set, in accordance with some embodiments described herein.

FIGS. 2A and 2B illustrate perspective and cross-sectional perspective views of the KVO infusion control device, in accordance with some embodiments described herein.

FIG. 3A illustrates a cross-sectional view of the KVO infusion control devic, in accordance with some embodiments disclosed herein.

FIG. 3B illustrates a close-up cross-sectional view of the first embodiment of the spiking system before a distilled water chamber is spiked, in accordance with some embodiments disclosed herein.

FIG. 3C illustrates a close-up cross-sectional view of the first embodiment of the spiking system when the distilled water chamber is spiked, in accordance with some embodiments disclosed herein.

FIGS. 4A and 4B illustrate cross-sectional views of the KVO infusion control device having a second embodiment of the spiking system, in accordance with some embodiments disclosed herein.

FIGS. 5A and 5B illustrate cross-sectional views of the KVO infusion control device having an alternative embodiment of the SAP component, in accordance with some embodiments disclosed herein.

FIGS. 6A-6B illustrate the function of various components of the KVO infusion control device, in accordance with some embodiments disclosed herein.

### DETAILED DESCRIPTION

In the following detailed description, numerous specific details are set forth to provide a full understanding of the subject technology. The subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

Maintaining the patency of an IV catheter is desirable in situations where a patient needs to receive medication through infusion on a repeated or intermittent basis. When maintaining the patency of an IV catheter, it is important to take steps to mitigate occlusions in the catheter line to avoid both the need to reinsert the catheter line in the patient's vein and infections in the patient's bloodstream. The following devices and methods provide design modifications to overcome the foregoing issues.

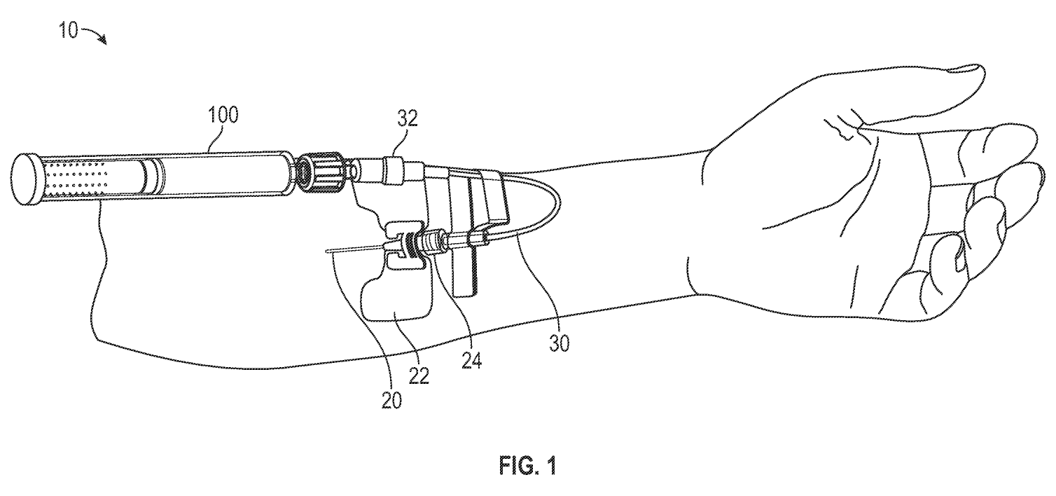

Referring now to the figures, FIG. 1 illustrates a KVO infusion control device being used in combination with an IV set, in accordance with some embodiments described herein. The IV set 10 is attached to the patient's arm and includes a catheter 20, a catheter hub 22, catheter wings 24, an extension tube 30, a fluid connector 32 (e.g., a needleless fluid connector or a female luer), and the KVO infusion control device 100. The catheter 20 is inserted in one of the patient's veins and is directly coupled to the catheter hub 22, which is secured to the patient's arm by the catheter wings 24. The extension tube 30 fluidly connects the catheter hub 22 to the needleless fluid connector 32. In turn, the needleless fluid connector 32 is coupled to the KVO infusion control device 100. Accordingly, the KVO infusion control device 100 is coupled to the patient's vein and delivers a controlled flow of fluid (e.g., saline) to the avoid occlusions in the catheter line while the patient's vein is open.

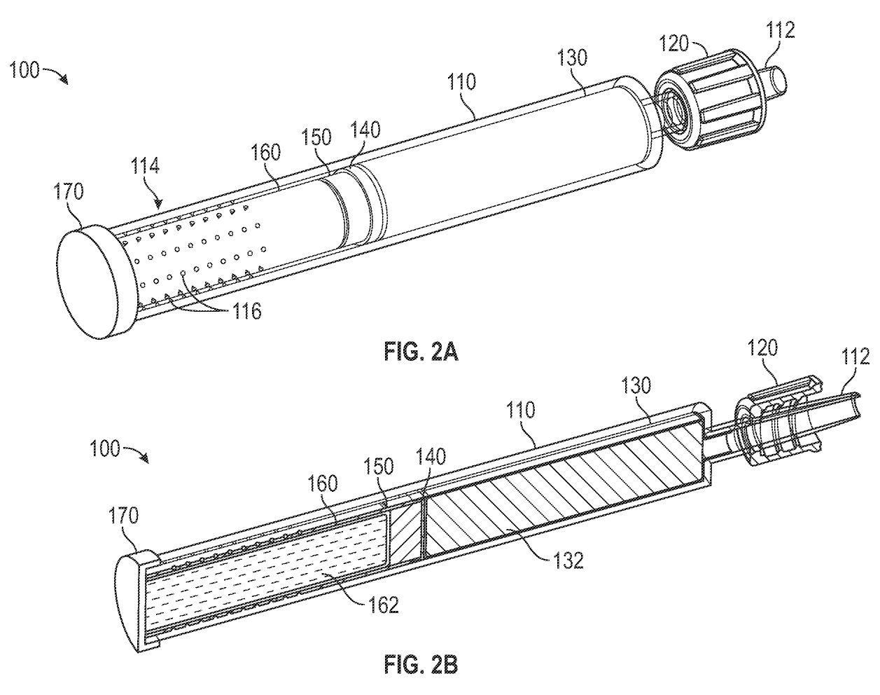

FIGS. 2A and 2B illustrate perspective and cross-sectional perspective views of the KVO infusion control device, in accordance with some embodiments described herein. The KVO infusion control device 100 comprises multiple components disposed within a housing 110. In particular, the housing 110 includes a flush chamber 130, a super absorbent polymer (SAP) pusher block 140, a SAP block 150, and a distilled water chamber 160. At a proximal end, the housing 110 has an opening that can be covered by a cap 170, and at a distal end, the housing 110 has a tip 112 that can be coupled to a male luer lock 120. The housing 110 also has a spiking system 114 that is configured to puncture the distilled water chamber 160. When the distilled water chamber 160 is punctured, distilled water 162 is released into the housing 110, and the distilled water 162 saturates the SAP block 150. When the SAP block 150 is wet, the SAP block 150 will expand inside the housing 110 and compress the flush chamber 130. As a result, the saline 132 inside the flush chamber 130 is distally advanced out of the distal tip 112 of the housing 110. The KVO infusion control device 100 facilitates a slow, continuous flow of saline 132 to the patient because the steady flow of distilled water 162 causes a steady expansion of the SAP block 150 and, thus, a steady displacement of the saline 132 out of the distal tip 112. As a result, saline 132 can be infused to the patient over a period of at least 8 hours. In some embodiments, the saline 132 can be infused to the patient over the course of approximately 10 hours.

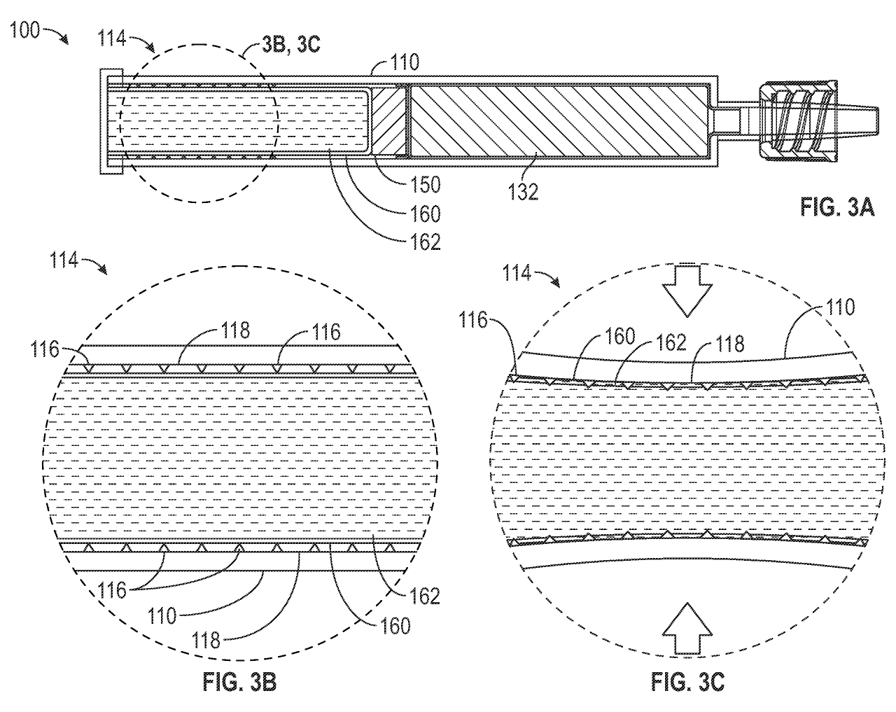

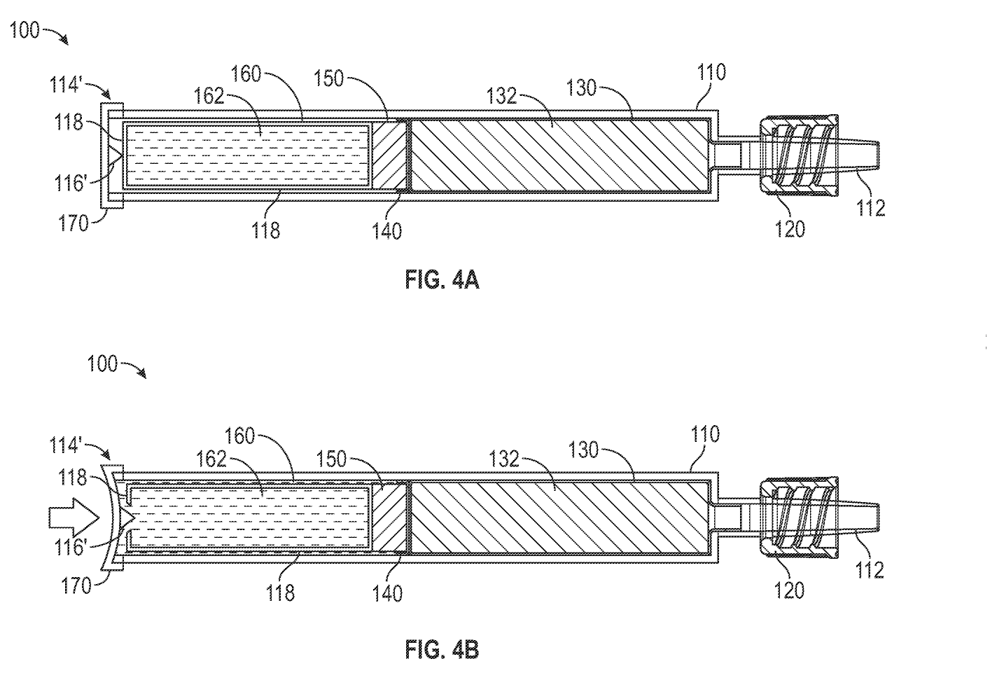

The housing 110 is a generally cylindrical tube with a tip 112 extending from the distal end of the housing 110. The tip 112 is configured to couple with a fluid connector (e.g., needleless fluid connector 32 shown in FIG. 1). The tip 112 can comprise a male luer lock 120, such as a standard male luer lock with a rotatable collar. Other types of fluid connectors are also possible. The housing 110 also has a spiking system 114 at a proximal portion of the cylindrical tube. As alluded to above, the spikes 116 of the spiking system 114 are configured to puncture the distilled water chamber 160 and facilitate delivery of the distilled water 162 to the SAP block 150. The structure and function of the spiking system 114 are discussed in greater detail below with respect to FIG. 3. Some embodiments of the KVO infusion control device 100 have a spiking system 114 at a proximal end of the housing 110. This embodiment is illustrated in FIGS. 4A and 4B and discussed in greater detail below.

The KVO infusion control device 100 also includes a flush chamber 130 disposed within a distal portion of the housing 110, adjacent to the tip 112. The flush chamber 130 is a generally cylindrical tube filled with saline 132. Optionally, the cylindrical tube of the flush chamber 130 is made of plastic or an elastomeric material. The flush chamber 130 fits inside the housing 110 such that an outer surface of the flush chamber 130 is in direct contact with an inner surface of the housing 110.

In some embodiments, the flush chamber 130 is configured to be compressed. When the flush chamber 130 is compressed, the saline 132 inside the flush chamber 130 is distally advanced out of the KVO infusion control device 100 via the distal tip 112 of the housing 110.

In other embodiments, the KVO infusion control device 100 has a flush region in the distal portion of the housing 110 instead of the flush chamber 130. In these embodiments, the saline 132 fills the distal portion of the housing 110 and is separated from the SAP block 150 by the uniform pusher 140. When the SAP block 150 expands, the SAP block 150 displaces the saline 132, and the saline 132 is released out of the KVO infusion control device 100 via the distal tip 112 of the housing 110.

The KVO infusion control device 100 also includes a SAP block 150 disposed within the housing 110 and proximal to the flush chamber 130. The SAP block 150 is configured to expand when it comes into contact with fluid (e.g., with distilled water 162 from the distilled water chamber 160). When the SAP block 150 expands, it pushes distally on the flush chamber 130, which drives the saline 132 inside the flush chamber 130 out of the KVO infusion control device 100 via the distal tip 112 of the housing 110. This effect is driven by osmosis. Because there are more ions in the SAP block 150 than in the distilled water 162, the molecules of the distilled water 162 flow into the SAP block 150 to establish equilibrium between the SAP block 150 and the distilled water 162 in the housing. In particular, the SAP block 150 can absorb the distilled water 162 at a rate of about 500 to 600 grams per gram.

The volume of the SAP block 150 increases due to the water, and, because the SAP block 150 is restrained by the inner surface of the housing 110 and the distilled water chamber 160, the SAP block expands distally when wet and advances the saline 132 towards the distal tip 112. The extent to which the volume of the SAP block 150 increases directly correlates with the amount of distilled water 162 to which the SAP block 150 is exposed. Accordingly, the rate at which the SAP block 150 advances the saline 132 out of the KVO infusion control device 100 can depend on the rate at which the distilled water 162 flows into the housing 110 and reaches the SAP block 150. This is described in greater detail below with respect to FIG. 3.

The rate at which the SAP block 150 advances the saline 132 out of the KVO infusion control device 100 can also depend on the material that makes up the SAP block 150. This is because the extent to which the SAP block 150 swells can depend on the crosslinking ability of the polymer that makes up the SAP block 150. Some embodiments of the KVO infusion control device 100 have a SAP block 150 made of sodium polyacrylate. Other embodiments have a SAP block 150 made of poly(acrylamide-co-acrylic acid) potassium salt. Both sodium polyacrylate and poly(acrylamide-co-acrylic acid) potassium salt swell in a range of about 270 to 300 grams per gram, which facilitates slow and consistent delivery of saline 132 to the patient.

The SAP block 150 has a generally cylindrical shape and fits inside the housing 110 between the flush chamber 130 and the distilled water chamber 160. The SAP block 150 can fit snugly inside the housing 110. Optionally, there is space (e.g., space 118 shown in FIG. 3) between the SAP block 150 and the inner surface of the housing 110.

In some embodiments, the KVO infusion control device 100 has a uniform pusher 140 that is positioned between the flush chamber 130 and the SAP block 150. When the SAP block 150 is exposed to the distilled water 162 and expands, the SAP block 150 applies a distal force to the uniform pusher 140. In turn, the uniform pusher 140 applies a distal force to the flush chamber 130 to drive the saline 132 out of the tip 112 of the KVO infusion control device 100.

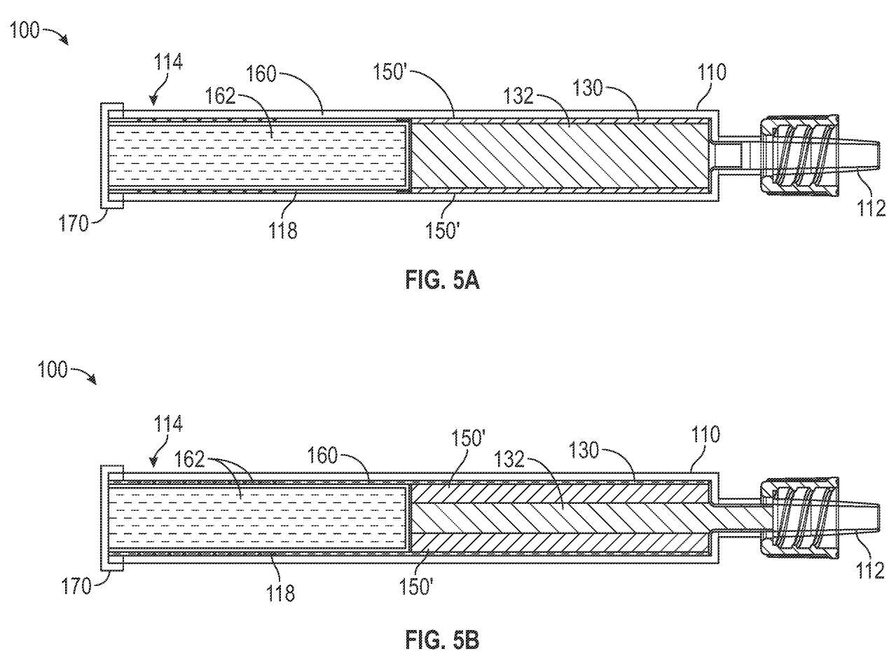

In some embodiments, the KVO infusion control device 100 has a SAP tube 150' instead of a SAP block 150. The SAP tube 150' is positioned along the inner surface of the flush chamber 130. This is described in greater detail below with respect to FIGS. 5A and 5B.

The proximal portion of the housing 110 is occupied by the distilled water chamber 160. The distilled water chamber 160 is positioned proximal to the SAP block 150. The proximal end of the housing 110 has an opening (not shown). In some embodiments, the distilled water chamber 160, which is filled with distilled water 162, is inserted into the housing 110 via the opening. In other embodiments, the distilled water chamber 160 remains inside the housing 110 and is refilled with distilled water 162 by pouring the distilled water 162 into the distilled water chamber 160 via the opening. The housing 110 can be closed using the cap 170, which can snap over the proximal end of the housing 110 and the proximal end of the distilled water chamber 160.

The distilled water chamber 160 is also adjacent to the spiking system 114. A caregiver can squeeze or push on the housing 110 or the cap 170 to spike the distilled water chamber 160 with the spiking system 114. When the distilled water chamber 160 is spiked, the distilled water 162 flows out of the distilled water chamber, into the housing 110, and towards the SAP block 150. This is described in greater detail with respect to FIG. 3.

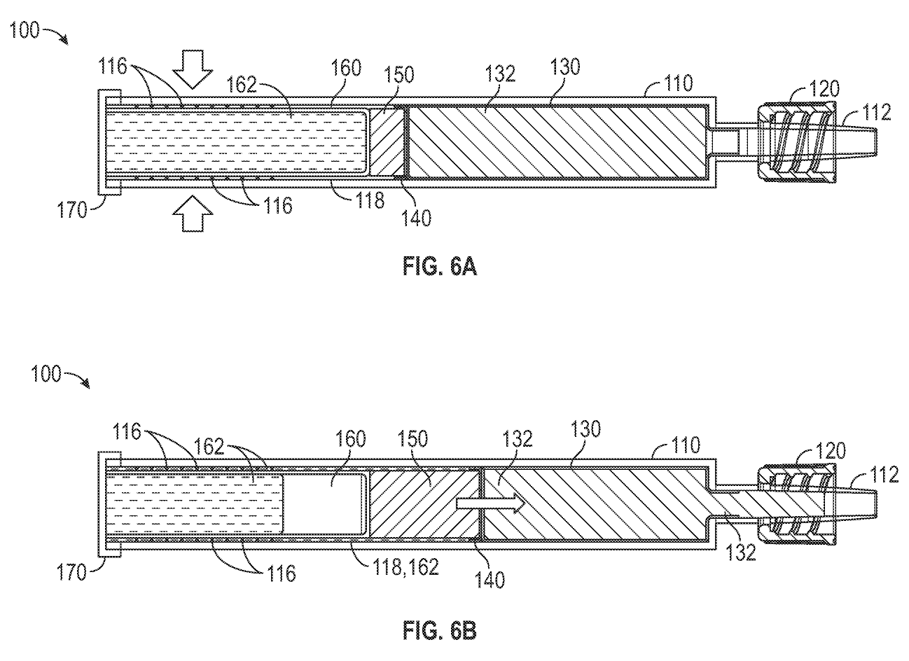

FIG. 3 illustrates a cross-sectional view of the KVO infusion control device and close-up cross-sectional views of a first embodiment of the spiking system, in accordance with some embodiments disclosed herein. The KVO infusion control device 100 shown in section A of FIG. 3 is configured to release distilled water 162 from the distilled water chamber 160 when the distilled water chamber 160 is spiked or punctured by the spiking system 114. When the distilled water chamber 160 is spiked, the distilled water 162 flows out of the distilled water chamber 160 and into the space 118 between an outer surface of the distilled water chamber 160 and the inner surface of the housing 110. This is shown in section C of FIG. 3. The distilled water 162 flows along the space 118 and towards the SAP block 150, which causes the SAP block 150 to expand. The expansion of the SAP block 150 is shown in FIGS. 6A-6B.

As shown in section B of FIG. 3, the spiking system 114 comprises one or more spikes 116 positioned on the inner surface of the housing. The spikes 116 protrude from the inner surface of the housing 110 such that the sharp ends point radially inward towards the outer surface of the distilled water chamber 160. The spikes 116 can be circumferentially arranged around the inner surface of the proximal portion of the housing 110. This is best seen in FIG. 2A. This arrangement of the spikes 116 can be ideal where the KVO infusion control device 100 is going to be laid horizontally on the patient's arm, as shown in FIG. 1. That is because the circumferential arrangement of the spikes 116 facilitates the flow of distilled water 162 out of the distilled water chamber 160 regardless of the rotational orientation of the KVO infusion control device 100 relative to the patient's arm. In some embodiments, the spikes 116 can be positioned circumferentially around the inner surface of the housing 110 at a location that is closer to the SAP block 150 (i.e., around a distal portion of the distilled water chamber 160). This can be beneficial in situations where the KVO infusion control device 100 can be expected to be tilted with the distal tip 112 slightly downwards (i.e., towards the ground) and a higher infusion rate of saline 132 is desirable because gravity will cause the distilled water 162 to rest in the distal portion of the distilled water chamber 160.

In the embodiment shown in FIGS. 1-3, the caregiver can spike the distilled water chamber 160 by squeezing the proximal portion of the housing 110 (i.e., pinching the housing 110 or applying a radially inward force to the housing 110). As shown in section C of FIG. 3, the inward force applied to the proximal portion of the housing 110 will drive the spikes 116 into the distilled water chamber 160. The sharp edges of the spikes 116 puncture the walls of the distilled water chamber 160. When the distilled water chamber 160 is spiked, the distilled water 162 flows out of the distilled water chamber 160 and around the spikes 116 via the holes formed by the spikes 116.

The quantity of the spikes 116 of the spiking system 114 can influence the rate at which the distilled water 162 reaches the SAP block 150. In turn, the quantity and placement of the spikes 116 influences the rate at which the SAP block 150 expands and, thus, the rate at which saline 132 is delivered to the patient. For example, increasing the number of spikes 116 increases the rate at which the distilled water 162 flows out of the distilled water chamber 160 and into the space 118 towards the SAP block 150 because there are more flow paths for the distilled water 162. Accordingly, increasing the number of spikes 116 increases the flow rate of saline 132 as the saline 132 is delivered to the patient. For the same reason, decreasing the number of spikes 116 decreases the rate at which the distilled water 162 reaches the SAP block 150 and, thus, the rate at which saline 132 is delivered to the patient.

Moreover, the size of the spikes 116 of the spiking system 114 can influence the rate at which the saline 132 is delivered to the patient. Smaller spikes 116 create smaller holes in the distilled water chamber 160. Consequently, distilled water 162 flows out of the distilled water chamber 160 more slowly when the spikes 116 are smaller, resulting in a slower infusion of saline 132 to the patient. Conversely, larger spikes puncture larger holes in the distilled water chamber 160, so saline 132 is delivered at a greater rate to the patient.

The size of the space 118 can limit the influence that the quantity and size of the spikes 116 can have on the flow rate of saline 132 out of the KVO infusion control device 100. Where the space 118 is smaller, the quantity and size of the spikes 116 has less influence on the flow rate of saline 132 because the smaller space 118 limits the rate at which the distilled water 162 reaches the SAP block 150. On the other hand, where the space 118 is larger, the quantity and size of the spikes 116 has more influence on the flow rate of saline 132. In some embodiments, the thickness of the space 118 is approximately 5% of the diameter of the housing 110, which means that approximately 80% of the housing 110 is occupied by the distilled water chamber 160.

FIGS. 4A and 4B illustrate cross-sectional views of the KVO infusion control device having a second embodiment of the spiking system, in accordance with some embodiments disclosed herein. FIG. 4A shows a KVO infusion control device 100 with a housing 100 having a distal tip 112 coupled to a male luer lock 120, a flush chamber 130 filled with saline 132, a uniform pusher 140 just proximal to the flush chamber 130, a SAP block 150 immediately adjacent to the uniform pusher 140, a distilled water chamber 160 filled with distilled water 162 and positioned immediately proximal to the SAP block, and a spiking system 114' at the proximal end of the housing 110. The proximal portion of the housing 110 has a space 118 arranged circumferentially between the inner surface of the housing 110 and the SAP block 150, the distilled water chamber 160, and the spiking system 114'. This space 118 can be filled with distilled water 162 when the distilled water chamber 160 is spiked by the spikes 116' of the spiking system 114'.

The spiking system 114' includes one or more spikes 116' positioned at the proximal end of the housing 110 with the sharp end of the spikes 116' being pointed at the distilled water chamber 160. In the embodiment shown, one spike 116' is coupled to the cap 170, and when a distal force is applied to the cap 170 (e.g., by a caregiver), the spike 116' is driven into the distilled water chamber 160, and distilled water 162 flows towards the SAP block 150. As described above with respect to the first embodiment of the spiking system, the SAP block 150 expands when it contacts water, which pushes the uniform pusher 140 into the flush chamber 130. The flush chamber 130 is compressed, and the saline 132 is squeezed out the distal tip 112 of the KVO infusion control device 100. In other embodiments, the spike 116' is coupled to the distilled water chamber 160 instead of the cap 170. Optionally, the spike 116' is coupled to the proximal end of the inner surface of the housing 110. Embodiments with the spike 116' (or spikes 116') at the proximal end of the KVO infusion control device 100 can be particularly useful where the KVO infusion control device 100 is expected to be positioned with the proximal end of the KVO infusion control device 100 downward (i.e., the cap 170 is pointed towards the floor) because gravity will cause the distilled water 162 to sit at the proximal end of the distilled water chamber 160, near the spikes 116'.

In some embodiments, the spiking system 114' comprises multiple spikes 116' instead of a single spike 116'. The multiple spikes 116' can be arranged near the radial center of the distilled water chamber 160 (i.e., approximately in line with the lumen of the distal tip 112). The multiple spikes 116' can also be arranged near the inner surface of the distilled water chamber 160. This embodiment can be particularly useful where the KVO infusion control device 100 is oriented horizontally (e.g., on the patient's arm, as shown in FIG. 1) because gravity will cause the distilled water 162 to sit on the lower part (i.e., closer to the ground) of the inner surface of the distilled water chamber 160.

Some embodiments of the KVO infusion control device 100 can include one or more spikes 116 around the proximal portion of the inner surface of the housing 110 (e.g., as shown in FIG. 3) in addition to one or more spikes 116' positioned at a proximal end of the distilled water chamber 160 (e.g., as shown in FIGS. 4A and 4B). In embodiments that have both spikes 116 and 116', the caregiver can spike the distilled water chamber 160 by applying one or both of a radially inward force to the proximal portion of the KVO infusion control device 100 and a distal force to the proximal end of the KVO infusion control device 100.

FIGS. 5A and 5B illustrate cross-sectional views of the KVO infusion control device having an alternative embodiment of the SAP component, in accordance with some embodiments disclosed herein. FIG. 5A shows the KVO infusion control device 100 before the distilled water chamber 160 is spiked by the spiking system 114. The KVO infusion control device 100 is made up of a housing 110 that holds a flush chamber 130 filled with saline 132 and a distilled water chamber 160 filled with distilled water 162. In this embodiment, the flush chamber 130 is adjacent to the distilled water chamber 160. This is because this embodiment has a SAP tube 150' instead of a SAP block 150, like the one shown in FIGS. 1-4B and 6A-6B. The SAP tube 150' is made of the same material as the SAP block 150 and serves a similar function (i.e., absorb distilled water 162 and expand to displace saline 132 out of the tip 112 of the housing 110) even though the SAP tube 150' is a cylindrical tube instead of a disc or cylindrical block.

In some embodiments, the SAP tube 150' lines the inner surface of the flush chamber 130. In other embodiments, the SAP tube 150' is the flush chamber 130-i.e., the inner surface of the flush chamber 130 is made of SAP material. In embodiments where the inner surface of the flush chamber 130 is made of SAP material, the SAP material is surrounded by a film that is permeable by the distilled water 162 (i.e., the outer surface of the flush chamber is made of a permeable film or material).

When the distilled water chamber 160 is spiked by the spiking system 114 (or 114'), the distilled water 162 enters the space 118 between the outer surface of the distilled water chamber 160 and the inner surface of the housing 110. The distilled water 162 reaches the flush chamber 130 and, thus, the SAP tube 150'. Upon contact with the distilled water 162, the SAP tube 150' expands. Because the SAP tube 150' is constrained by the housing 110 on the distal side of the SAP tube 150' and by the distilled water chamber 160 on the proximal side of the SAP tube 150', the SAP tube 150' expands radially inward. This is shown in FIG. 5B. As a result, the expanded SAP tube 150' displaces the saline 132, and the saline 132 is pushed out the distal tip 112 of the housing. When the KVO infusion control device 100 is coupled to a catheter line (e.g., via the connector 32 shown in FIG. 1), the saline 132 is delivered to the patient. (Notably, the saline 132 does not cause the SAP tube 150' to expand meaningfully because the difference in ions within the polymer matrices of saline 132 and the SAP tube 150' is small.)

The present disclosure also includes methods for using the KVO infusion control device. Accordingly, FIGS. 6A-6B illustrate the function of various components of the KVO infusion control device, in accordance with some embodiments disclosed herein. After the KVO infusion control device 100 is connected to the patient's catheter line (i.e., the male luer lock 120 is coupled to a needleless fluid connector), the caregiver pinches or presses the proximal portion of the housing 110 (as indicated by the arrows in FIG. 6A) to drive the spikes 116 into the distilled water chamber 160.

When the spikes 116 puncture the distilled water chamber 160, the distilled water 162 will flow out of the distilled water chamber (via the puncture holes formed by the spikes 116) and into the space 118 between the outer surface of the distilled water chamber 160 and the inner surface of the housing 110. This is shown in FIG. 6B. FIG. 6B also shows that the distilled water 162 flows until it surrounds the SAP block 150. When the distilled water 162 comes into contact with the SAP block 150, the distilled water 162 flows into the SAP block 150 due to osmosis. The SAP block 150 absorbs the distilled water 162 and gradually increases in volume. The arrow in FIG. 6B illustrates that the SAP block 150 pushes on the uniform pusher 140. The SAP block 150 applies a distal force to the uniform pusher 140 (or, where there is no uniform pusher 140, directly to the flush chamber 130) because the SAP block 150 is constrained on its proximal side by the distilled water chamber 160. The distal force from the SAP block 150 gradually compresses the flush chamber 130 and distally advances the saline 132 that is inside the flush chamber 130. As a result, a steady and continuous flow of saline 132 is delivered to the patient and the caregiver can keep the catheter line open with a significantly reduced risk of occlusions.

When the flush chamber 130 is empty, the KVO infusion control device 100 can be disconnected from the patient's catheter line so that the flush chamber 130 can be replenished with saline 132.

When the distilled water chamber 160 is empty, the cap 170 can be removed to expose the opening at the proximal end of the housing 110. In some embodiments, the empty distilled water chamber 160 can be removed from the housing 110 and replaced with a distilled water chamber 160 that is full of distilled water 162. In other embodiments, the caregiver can pour distilled water 162 into the distilled water chamber 160 (while the distilled water chamber 160 is still inside the housing) via the openings at the proximal ends of the housing 110 and the distilled water chamber 160. After the distilled water chamber 160 is replaced or the distilled water 162 is refilled, the caregiver can place the cap 170 back on the housing 110 and spike the distilled water chamber 160 with the spikes 116 to continue delivering the saline 132 to the patient.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 9, or clause 16. The other clauses can be presented in a similar manner.

Clause 1. A keep vein open (KVO) infusion control device, the KVO infusion control device comprising: a housing comprising a tube having a tip at a distal end of the tube and a spiking system at a proximal portion of the tube; a flush chamber disposed within a distal portion of the housing, the flush chamber configured to be filled with saline; a super absorbent polymer (SAP) block disposed within the housing and proximal to the flush chamber, the SAP block configured to expand when in contact with distilled water; and a distilled water chamber disposed within the housing adjacent to the spiking system and proximal to the SAP block, the distilled water chamber configured to be filled with the distilled water, wherein the spiking system is configured to facilitate delivery of the distilled water to the SAP block, wherein the SAP block is configured to compress the flush chamber and distally advance the saline out of the tip when the SAP block expands.

Clause 2. The KVO infusion control device of Clause 1, wherein the tip is configured to couple with a needleless fluid connector.

Clause 3. The KVO infusion control device of Clause 1, wherein the tip comprises a male luer lock.

Clause 4. The KVO infusion control device of Clause 1, wherein the SAP block is generally cylindrical.

Clause 5. The KVO infusion control device of Clause 1, wherein the SAP block comprises sodium polyacrylate.

Clause 6. The KVO infusion control device of Clause 1, wherein the SAP block comprises poly (acrylamide-co-acrylic acid) potassium salt.

Clause 7. The KVO infusion control device of Clause 1, further comprising a uniform pusher positioned between the SAP block and the flush chamber.

Clause 8. The KVO infusion control device of Clause 7, wherein the uniform pusher is configured to receive a force from the SAP block as the SAP block expands and apply the force to the flush chamber to distally advance the saline out of the tip.

Clause 9. The KVO infusion control device of Clause 1, wherein the distilled water chamber is configured to be refilled with the distilled water via an opening at a proximal end of the distilled water chamber.

Clause 10. The KVO infusion control device of Clause 1, further comprising a cap coupled to a proximal end of the housing, wherein the cap is configured to close the distilled water chamber.

Clause 11. The KVO infusion control device of Clause 1, wherein the spiking system comprises one or more spikes positioned on an inner surface of the housing, each of the one or more spikes comprising a sharp end configured to puncture the distilled water chamber to fluidly connect the distilled water chamber to the housing.

Clause 12. The KVO infusion control device of Clause 1, wherein the spiking system comprises one or more spikes configured to puncture the distilled water chamber and fluidly connect the distilled water chamber to the SAP block.

Clause 13. The KVO infusion control device of Clause 1, wherein the spiking system is configured to puncture the distilled water chamber and release the distilled water into the housing.

Clause 14. A keep vein open (KVO) infusion control device, the KVO infusion control device comprising: a housing comprising a tube having a distal tip; a flush chamber disposed within a distal portion of the housing, the flush chamber configured to be filled with saline; a super absorbent polymer (SAP) block disposed within the housing and proximal to the flush chamber, the SAP block configured to expand when in contact with distilled water; a distilled water chamber disposed within the housing and proximal to the SAP block, the distilled water chamber configured to be filled with the distilled water; and a spiking system positioned on an inner surface of the housing and proximal to the distilled water chamber, the spiking system configured to fluidly connect the distilled water chamber to the housing and facilitate delivery of the distilled water to the SAP block, wherein the SAP block is configured to compress the flush chamber and distally advance the saline out of the distal tip when the SAP block expands.

Clause 15. The KVO infusion control device of Clause 14, further comprising a cap coupled to a proximal end of the housing, wherein the cap is configured to close the distilled water chamber.

Clause 16. The KVO infusion control device of Clause 15, wherein the spiking system is attached to the cap and configured to puncture the distilled water chamber when the cap is coupled to the proximal end of the housing.

Clause 17. The KVO infusion control device of Clause 14, wherein the spiking system comprises one or more spikes, each of the one or more spikes comprising a distally oriented sharp end configured to puncture the distilled water chamber to fluidly connect the distilled water chamber to the housing.

Clause 18. A keep vein open (KVO) infusion control device, the KVO infusion control device comprising: a housing comprising a tube having a tip at a distal end of the tube and a spiking system at a proximal portion of the tube; a flush chamber disposed within a distal portion of the housing and configured to be filled with saline, the flush chamber comprising a super absorbent polymer (SAP) tube that is configured to expand when in contact with distilled water; and a distilled water chamber disposed within the housing adjacent to the spiking system and proximal to the flush chamber, the distilled water chamber configured to be filled with the distilled water, wherein the spiking system is configured to facilitate delivery of the distilled water to the SAP tube, wherein the SAP tube is configured to displace the saline out of the tip when the SAP tube expands.

Clause 19. The KVO infusion control device of Clause 18, wherein the spiking system is configured to puncture the distilled water chamber and release the distilled water into the housing so that the distilled water can contact the SAP tube.

Clause 20. The KVO infusion control device of Clause 18, wherein, when in contact with the distilled water, the SAP tube is configured to expand radially inward and displace the saline out of the tip.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean "one and only one" unless specifically so stated, but rather "one or more." Unless specifically stated otherwise, the term "some" refers to one or more. Pronouns in the masculine (e.g., his) include the feminine and neuter gender (e.g., her and its) and vice versa. Headings and subheadings, if any, are used for convenience only and do not limit the invention.

The word "exemplary" is used herein to mean "serving as an example or illustration." Any aspect or design described herein as "exemplary" is not necessarily to be construed as preferred or advantageous over other aspects or designs. In one aspect, various alternative configurations and operations described herein may be considered to be at least equivalent.

A phrase such as an "aspect" does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an "embodiment" does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a "configuration" does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term "coupled" or the like may refer to being directly coupled. In another aspect, the term "coupled" or the like may refer to being indirectly coupled.

Terms such as "top," "bottom," "front," "rear," and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase "means for" or, in the case of a method claim, the element is recited using the phrase "step for." Furthermore, to the extent that the term "include," "have," or the like is used, such term is intended to be inclusive in a manner similar to the term "comprise" as "comprise" is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. §101, 102, or 103, nor should they be interpreted in such a way.

What is claimed is:

1. A keep vein open (KVO) infusion control device, the KVO infusion control device comprising:
a housing comprising a tube having a tip at a distal end of the tube and a spiking system at a proximal portion of the tube;
a flush chamber disposed within a distal portion of the housing, the flush chamber configured to be filled with saline;
a super absorbent polymer (SAP) block disposed within the housing and proximal to the flush chamber, the SAP block configured to expand when in contact with distilled water; and
a distilled water chamber disposed within the housing adjacent to the spiking system and proximal to the SAP block, the distilled water chamber configured to be filled with the distilled water,
wherein the spiking system is configured to facilitate delivery of the distilled water to the SAP block,
wherein the SAP block is configured to compress the flush chamber and distally advance the saline out of the tip when the SAP block expands.

2. The KVO infusion control device of claim 1, wherein the tip is configured to couple with a needleless fluid connector.

3. The KVO infusion control device of claim 1, wherein the tip comprises a male luer lock.

4. The KVO infusion control device of claim 1, wherein the SAP block is generally cylindrical.

5. The KVO infusion control device of claim 1, wherein the SAP block comprises sodium polyacrylate.

6. The KVO infusion control device of claim 1, wherein the SAP block comprises poly(acrylamide-co-acrylic acid) potassium salt.

7. The KVO infusion control device of claim 1, further comprising a uniform pusher positioned between the SAP block and the flush chamber.

8. The KVO infusion control device of claim 7, wherein the uniform pusher is configured to receive a force from the SAP block as the SAP block expands and apply the force to the flush chamber to distally advance the saline out of the tip.

9. The KVO infusion control device of claim 1, wherein the distilled water chamber is configured to be refilled with the distilled water via an opening at a proximal end of the distilled water chamber.

10. The KVO infusion control device of claim 1, further comprising a cap coupled to a proximal end of the housing, wherein the cap is configured to close the distilled water chamber.

11. The KVO infusion control device of claim 1, wherein the spiking system comprises one or more spikes positioned on an inner surface of the housing, each of the one or more spikes comprising a sharp end configured to puncture the distilled water chamber to fluidly connect the distilled water chamber to the housing.

12. The KVO infusion control device of claim 1, wherein the spiking system comprises one or more spikes configured to puncture the distilled water chamber and fluidly connect the distilled water chamber to the SAP block.

13. The KVO infusion control device of claim 1, wherein the spiking system is configured to puncture the distilled water chamber and release the distilled water into the housing.

14. A keep vein open (KVO) infusion control device, the KVO infusion control device comprising:
a housing comprising a tube having a distal tip;
a flush chamber disposed within a distal portion of the housing, the flush chamber configured to be filled with saline;
a super absorbent polymer (SAP) block disposed within the housing and proximal to the flush chamber, the SAP block configured to expand when in contact with distilled water;
a distilled water chamber disposed within the housing and proximal to the SAP block, the distilled water chamber configured to be filled with the distilled water; and
a spiking system positioned on an inner surface of the housing and proximal to the distilled water chamber, the spiking system configured to fluidly connect the distilled water chamber to the housing and facilitate delivery of the distilled water to the SAP block,
wherein the SAP block is configured to compress the flush chamber and distally advance the saline out of the distal tip when the SAP block expands.

15. The KVO infusion control device of claim 14, further comprising a cap coupled to a proximal end of the housing, wherein the cap is configured to close the distilled water chamber.

16. The KVO infusion control device of claim 15, wherein the spiking system is attached to the cap and configured to puncture the distilled water chamber when the cap is coupled to the proximal end of the housing.

17. The KVO infusion control device of claim 14, wherein the spiking system comprises one or more spikes, each of the one or more spikes comprising a distally oriented sharp end configured to puncture the distilled water chamber to fluidly connect the distilled water chamber to the housing.

18. A keep vein open (KVO) infusion control device, the KVO infusion control device comprising:
a housing comprising a tube having a tip at a distal end of the tube and a spiking system at a proximal portion of the tube;
a flush chamber disposed within a distal portion of the housing and configured to be filled with saline, the flush chamber comprising a super absorbent polymer (SAP) tube that is configured to expand when in contact with distilled water; and
a distilled water chamber disposed within the housing adjacent to the spiking system and proximal to the flush chamber, the distilled water chamber configured to be filled with the distilled water,
wherein the spiking system is configured to facilitate delivery of the distilled water to the SAP tube,
wherein the SAP tube is configured to displace the saline out of the tip when the SAP tube expands.

19. The KVO infusion control device of claim 18, wherein the spiking system is configured to puncture the distilled water chamber and release the distilled water into the housing so that the distilled water can contact the SAP tube.

20. The KVO infusion control device of claim 18, wherein, when in contact with the distilled water, the SAP tube is configured to expand radially inward and displace the saline out of the tip.
